(19) United States
(12) Patent Application Publication
(10) Pub. No.: US 2004/0067267 A1
(43) Pub. Date: Apr. 8, 2004

(54) Process for preparation of cholesterol lowering compositions from garlic

(76) Inventors: Pothapragada Suryanarayana Murthy, Noida (IN); Patti Ratnakar, Clifton Heights, PA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/270,742

(22) Filed: Oct. 11, 2002

Related U.S. Application Data
(60) Provisional application No. 60/417,414, filed on Oct. 8, 2002.

(51) Int. Cl. A61K 35/78

(52) U.S. Cl. 424/754

(57) ABSTRACT

The present invention is directed in part towards methods of preparing a blood cholesterol-lowering extract from garlic, method of treating a mammal with a high blood cholesterol level using a garlic extract, and pharmaceutical compositions comprising garlic extracts.

### RELATED APPLICATIONS

This application claims priority to Provisional Application Serial No. ______ entitled PROCESS FOR PREPARATION OF CHOLESTEROL LOWERING COMPOSITIONS FROM GARLIC filed on Oct. 8, 2002. The subject matter of the aforementioned application is hereby incorporated herein by reference in its entirety.

### BACKGROUND OF THE INVENTION

It is estimated that cardiovascular diseases (CVD) account for major proportion (23%) of all the deaths at global level. In developing countries CVD accounts for 16% of the total deaths (World Health Organization technical report, 1990). Deaths due to CVD are fast increasing in developing countries too. Atherosclerosis together with its other complications, is the pathological process that underlies most cases of coronary heart diseases (CHD). It is also responsible for thrombotic and embolic strokes, aortic aneurymal disease, renovascular hypertension, peripheral vascular diseases and other clinical syndromes. Therefore, prevention of atherosclerosis is of paramount importance for increasing the human life span and for better health.

Etiological factors: The dietary habits of people contribute to the increased incidence of coronary and other atherosclerotic diseases in several ways. High intake of saturated fat and cholesterol (Stamler & Shekelle, 1988) along with high calories and consequent obesity, as well as low fibre intake lead to high levels of total serum cholesterol and its atherogenic subfractions (low density lipoproteins (LDL) and very low density lipoproteins (VLDL)) and to high prevalence and incidence rates of hypercholesterolemia from childhood and youth onwards. Increased serum cholesterol is one of the major etiologically significant risk factors for CHD and other atherosclerotic diseases. Excess lipoprotein (a), a cholesteryl ester rich lipoprotein with LDL like core to which apolipoprotein (a) is linked is also a strong and independent risk factor for atherosclerotic disease (Uterman 1994).

Treatment of Coronary Heart Diseases

Dietary modifications: Individuals with hyperlipidemias especially hyperlipoproteinemias are recommended diet containing low cholesterol and saturated animal fat and relatively higher polyunsaturated vegetable oils in order to prevent increase in serum LDL and VLDL levels.

Elimination of other risk factors: Hyperlipoproteinemia is exacerbated by some other diseases like diabetes mellitus, alcoholism, hypothyroidism, nephrotic syndrome, acute renal failure, and use of oral contraceptives. The aim is the effective control of the exacerbating diseases.

Treatment with drugs: The therapy involves the administration of drugs that lower the elevated lipoproteins, either by decreasing their production or by increasing their removal from the circulation. Combination of these drugs has been successfully used in lowering LDL concentrations in patients with heterozygous familial hypercholesterolemia (Kane et al., 1981).

Nicotinic acid reduces triglyceride levels rapidly, when given in large doses, due to decrease in VLDL fraction of lipoproteins. It also causes mild to moderate increase in HDL (Carlson and Olsson 1979, Kane et al 1981). Clofibrate (ethyl ester of p-chlorophenoxy isobutyric acid) is used exclusively to treat familial dysbetali poproteinemia. It brings about mild change in the levels of cholesterol and moderate decrease in the levels of triglycerides in patients. Gemfibrozil (structurally related to clofibrate) is effective in reducing VLDL levels in patients not responding to diet.

Probucol is a sulfur-containing bis-phenol which decreases serum cholesterol LDL levels. However it also lowers HDL levels, thus limiting its use.

Effect on serum lipids: Since bile acids are required for the intestinal absorption of cholesterol, there is fecal loss of neutral sterols. Cholestyramine and colestipol are anion exchange resins which bind in bile acids and remove them from hepatic circulation and increase their fecal excretion. This in turn results in excretion of dietary cholesterol. Both the resins reduce cholesterol by lowering LDL levels. They show maximum effect within two weeks of therapy. They are very useful particularly in treating hypercholesterolemia. They do not have any beneficial effect on triglycerides. The homozygous familial hypercholesterolemic patients who totally lack in LDL receptors, do not respond to this therapy.

3-Hydroxy 3-methyl glutaryl (HMG) CoA reductase inhibitors: Compaction and mevinolin structurally resemble HMG CoA, and inhibit (competitively) HMG CoA reductase activity. They lower (30%) plasma LDL cholesterol levels. Given along with bile acid binding resin, these drugs decrease LDL levels by 50% (Bilheimer et al 1983). They did not show any effect on triglyceride and HDL levels. They are not administered to pregnant women since, HMG CoA reductase plays a crucial role in providing cholesterol and other non sterol compounds to developing fetus.

Neomycin is a second line drug for use in patients with primary hypercholesterolemia who are unable to use bile acid sequestrants. It reduced LDL cholesterol by 15-20% (Hoeg et al. 1984). Neomycin administration increases the fecal excretion of neutral steroids, but does not change bile acid excretion. d-Thyroxine, the optical isomer of the hormone L-thyroxine, has modest hypocholesterolemic activity. d-Thyroxine lowers LDL levels by increasing hepatic LDL receptor synthesis and thus LDL uptake by them (Thompson et al. 1981). However, it causes mild hyperthyroidism. Beta-Sitosterol, a plant sterol structurally similar to cholesterol (ethyl group at C24) lowers plasma LDL-cholesterol but does not show any effect on triglycerides. It may be acting by inhibiting absorption of dietary cholesterol (Kane and Malloy, 1982). Simvastatin reduces total cholesterol and LDL-C but its triglyceride reducing effect is slight (Current Ther. Res. 1996, 57, 418-419).

### SUMMARY OF THE INVENTION

Out of the drugs mentioned above, nicotinic acid can reduce only triglycerides level. Clofibrate is for dysbetalipoproteinemia. Probucol has the limitation of reducing HDL also. The bile acid binding resins (cholistyrine and colestipol) reduce cholesterol but not triglycerides and not useful in those lacking LDL receptors. The HMG CoA reductase inhibitor compactin and mevinolin reduce cholesterol but have no effect on triglyceride and HDL levels and cannot be used for pregnant women. The other drugs neomycin, d-thyroxine and beta sitosterol also have limited use.

There has been renewed interest in plant drugs because of their safety. There are many plant drugs known to reduce serum and tissue cholesterol and triglyceride levels. In fact one drug called Guggulu lipid from commiphora mukul has been developed in India from an indigenous plant as a hypocholesterolemic drug. Among the many plant drugs garlic is well known for its cholesterol lowering property.

Thus, in the first aspect, the invention is related to a method of preparing a blood cholesterol-lowering extract from garlic comprising: contacting crushed garlic with an alcohol to create a first mixture having a solid phase and a liquid phase; separating said solid phase of said first mixture from said liquid phase of said first mixture; contacting, at least once, said separated and concentrated liquid phase of said first mixture with chloroform to create a second mixture having an aqueous alcohol phase and a chloroform phase; separating said chloroform phase of said second mixture from said aqueous alcohol phase of said second mixture; concentrating said chloroform phase of said second mixture; and purifying said concentrated chloroform phase by chromatography and obtaining a light yellow product.

The invention also relates to a method of treating a mammal with a high blood cholesterol level comprising: identifying a mammal in need of such treatment; providing a garlic extract obtained according to claim 1; and administering said garlic extract to said mammal. Furthermore, the invention relates to a pharmaceutical composition comprising a garlic extract obtained according to claim 1, or a pharmaceutically acceptable salt thereof, and a pharmaceutically acceptable diluent, excipient, stabilizer or carrier.

### DETAILED DESCRIPTION OF THE PREFERRED EMBODIMENTS

Garlic is the most studied plant (bulb) for its beneficial hypocholesterolemic effects. The work done so far indicates certain aspects regarding garlic action, which include: favorable effects of reduction in serum and tissue lipids; no change in serum and tissue lipids; active garlic compounds and even whole garlic for a longer time of two months or more have to be administered in higher doses and therefore produce undesirable side effects; the garlic compounds are unstable in nature; and its strong odor makes it unacceptable to many persons.

Favorable Effects.

Whole Garlic

Raw garlic (25 g/days) along with 0.5 g cholesterol (Jain 1976) in diet (The diet is therefore atherogenic), 5 g fresh garlic bulbs per day with atherogenic diet for 7 days (Chang and Johnson 1980) and freeze dried powder along with atherogenic diet (Kamanna and Chandrasekhara (1982) were found to reduce serum and tissue cholesterol and other lipids to much lower levels than in the untreated controls. Studies of Chang and Johnson (1980) indicated that garlic inhibits the incorporation of 14C acetate and 14C sucrose into tissue lipids.

In human volunteers, Bordia and Bansal (1973) have observed that administration of 50 gm of garlic or its equivalent essential oil has counteracted the changes in blood lipids caused by ingestion of fat.

The epidemiological study of high significance by Sainani et. al. (1979) has revealed that those who consume high amount of garlic and onion in the diet have less serum cholesterol and triglyceride levels than those who consume less or totally abstain from them. In some studies conducted in patients with coronary heart disease, garlic administration has shown beneficial effect. A disturbing feature of garlic effect is that the fall in serum cholesterol level seen in 5 hypercholesterolemic patients given fresh juice of garlic 5 ml/kg/day for 2 months has not persisted after withdrawal of garlic treatment (Augusti 1977). Bordia (1981) has observed that patients suffering from coronary heart disease when given essential oil of garlic for 10 months, have shown steady decrease in LDL and VLDL along with increase in HDL levels.

Studies with Garlic Products

Allicin (100 mg/kg/day), obtained from the steam distillate of garlic, when fed for two months to normal rats could bring about decrease in total lipids, phospholipids, triglycerides and cholesterol levels both in serum and liver (Augusti & Mathew, 1974).

Essential oil from garlic could decrease serum cholesterol in rabbits fed cholesterol supplemented diet (Bordia et al. 1975) and inhibit the development of atherosclerotic lesions in the arteries by reducing serum cholesterol and phospholipid levels (Jain & Konar 1976, 1978). In an interesting experiment, Qureshi et al. (1983 a&b) have found that the water, methanol and petroleum ether soluble fractions of garlic have decreased the serum cholesterol and triglyceride levels in chicken and inhibited the hepatic HMG CoA reductase, fatty acid synthetase and 7 α-hydroxylase activity. Garlic is rich in sulfur containing compounds. Many sulfur containing amino acids have been isolated and tested for their effect on hypercholesterolemia. Out of these, S-allyl cysteine sulfoxide and S-methyl cysteine sulfoxide have shown hypolipidemic activity (Isokawa et al. 1973). They could prevent the increase in serum and liver cholesterol in rats fed cholesterol rich diet.

Garlic has no Effect

There are however also reports which contradict the above favorable changes by garlic administration. Arora & Arora (1981) have noted that administration of garlic oil along with fat to normal subjects has no favorable response either in serum lipid constituents like cholesterol, triglycerides and LDL-cholesterol or coagulation time and fibrinolytic activity. The same authors (Arora. et al. 1981) could not find any significant changes in blood lipids of both normal subjects and patients with ischemic heart disease after treatment with essential oil of garlic (3.75 mg/day) for 12 weeks. On the other hand the side effects like vomiting, diarrhea, anorexia, and weight loss have been observed. However, the reason for the negative findings is thought to be the lower dose of oil in this study.

Adverse Effects of Garlic

The raw form of garlic and higher doses of essential oil of garlic have many undesirable effects on animals and human subjects. They cause anemia, weight loss and irritation of digestive mucosa (Nakagawa, et. al. 1980). There are also some reports of severe allergic response, though rare, to garlic (van Ketel and de Haan, 1978 Papageorgiou et al. 1983).

Imada (1990) has recently investigated short term (3, 7 & 21 days) toxicity in rats. Oral administration of raw garlic juice or allicin has given rise to extensive edema, bleeding and ulceration of forestomach mucosa, depression of body weight gain, reduction in RBC count and hematocrit and increase of reticulocytes. In acute toxicological studies on mice oral LD 50 values (mg/kg b.wt) for some compounds present in garlic are as follows diallyldisulfide (male (m) 145, female (f) 130), allicin (m:309, f: 363); s-allyl mercapto cysteine (m:600; f: 922); diallyl sulfide (m: 2029; f:1805), s-allyl-L-cysteine (m:8890; f:9390).

Odor Modified Garlic

The characteristic strong and unpleasant odor of garlic remains the most unacceptable factor for many people. Some Japanese workers (Nagai & Osawa, 1974) have overcome this problem by subjecting the garlic to the ancient practice of ‘aging’ and curing in vogue in their country. Even after the removal of odor, the aged garlic retains the activity of reducing serum cholesterol levels. It is marketed under the trade name ‘Kyolic’ by M/s.Wakunaga of California, USA. Recently Lau et al. (1987) have carried out systematic studies showing that odor modified garlic extract brings down significantly serum cholesterol and triglyceride levels in human subjects. However, some of the findings are interesting. During the first two months period, cholesterol and triglyceride levels have actually increased, but significant decrease in their levels has been seen only after prolonged administration beyond two months. This kind of observation in patients fed garlic has been reported by Bordia (1981) who postulated that lipids are mobilized from tissues into blood. Chang & Johnson (1980) also noticed marginal increase in serum cholesterol and triglycerides, after 18 days administration of ethanolic extract of garlic to rats. Our studies in rabbits also gave similar results but in whole garlic (Chang & Johnson 1980) garlic juice (Jain & Vyas 975) and the extract of garlic (Brahmachari and Augusti 1962) and allicin (from steam distillation of garlic, Mathew and Augusti 1973) have been reported to bring down blood glucose levels by increasing serum insulin levels in human and animals.

Our Product from Garlic and its Advantages

Voluminous literature on garlic shows that by and large whole garlic and its preparations are undoubtedly useful as drugs for lowering cholesterol and other lipids in serum and tissues as required in coronary heart diseases. But the limitations are: (i) high doses to be used for several months (ii) toxic effects due to the requirement of high doses for a long time (iii) unstable nature of the preparations and (iv) strong odor.

We purified from garlic a compound which is active at a dose much lower than that of any other preparation reported so far. In view of this we did not find any toxic effects when fed to rabbits for six months.

Even though the preparation has slight garlic odor, it is much less than that of whole garlic or garlic oil.

We stabilized it by adding ascorbic acid 100 mg and vitamin E 50 mg, which prevent oxidation of the sulfur compounds of garlic. It is to be noted that the vitamins C and E are added at their daily recommended doses. Vitamin E is also useful as an antioxidant because lipid peroxidation products are known to increase in atherosclerosis and other related vascular disorders.

### EXAMPLES

### Example 1

### List of Raw Materials, Chemicals, Utilities

Garlic, absolute alcohol (ethanol) chloroform, silica gel G sodium sulphate (anlydrous), iodine, conc. Sulphuric acid, cholesterol, Kits for estimation of cholesterol, LDL VLDL and HDL Cholesterol and triacylglycerols, Muslin cloth, Whatman No. 1 filler paper.

### Example 2

### List of Equipment

Homogenizer, Rotary vacuum evaporator, Glass plates for thin layer chromatography, Cold room (4° C.), Deep freeze, Nitrogen cylinder.

### Example 3

### Preparation of Hypolipidemic Compound

Step 1

Preparation of ethanol extract: Garlic cloves (100 gm) devoid of dry skin were crushed in Waring blender and mixed thoroughly with 200 ml of absolute alcohol. The mixture was allowed to stand overnight in refrigerator and filtered through several layers of muslin cloth. The residue was reextracted with 200 ml of fresh ethanol and combined filtrate was filtered through Whatman no. 1 filter paper to remove any finely suspended particles. The filtrate was concentrated in a rotary vacuum evaporator at 30° C.

Step 2

Extraction with Chloroform: Residue after removal of alcohol from alcohol extract was extracted with chloroform (once with 50 ml and twice with 25 ml for every 100 ml of concentrate) in a separating funnel. The combined chloroform extracts were dried over anhydrous sodium sulfate for one hour in the cold and evaporated in vacuum at 30° C. The oily substance was dissolved in alcohol and stored under nitrogen at 4° C.

Step 3

Thin layer chromatographic purification: A slurry of silica gel G was made in distilled water (1:2 w/v) avoiding air bubbles and coated on 2 mm thick glass plates (20 cm×20 cm) forming a layer of 0.25 mm thickness. The plates were allowed to dry at room temperature and activated at 120° C. for 45 minutes before use.

The concentrated chloroform extract from Step 2 (50 μl) was applied on the plates and separated using the solvents chloroform:methanol 2:1 v/v). Plate was exposed to iodine vapours or sprayed with 50% H₂SO₄ and heated at 120° C. for 30 minutes to visualize the separated compounds. In Industrial scale separation this step is to be replaced by column chromatography using silica gel and eluting it with the solvent mixture. The procedure is shown Flow Chart 1. The active compound was extracted. Its structure is not known. It could be a sulfur containing compound related to allicin.

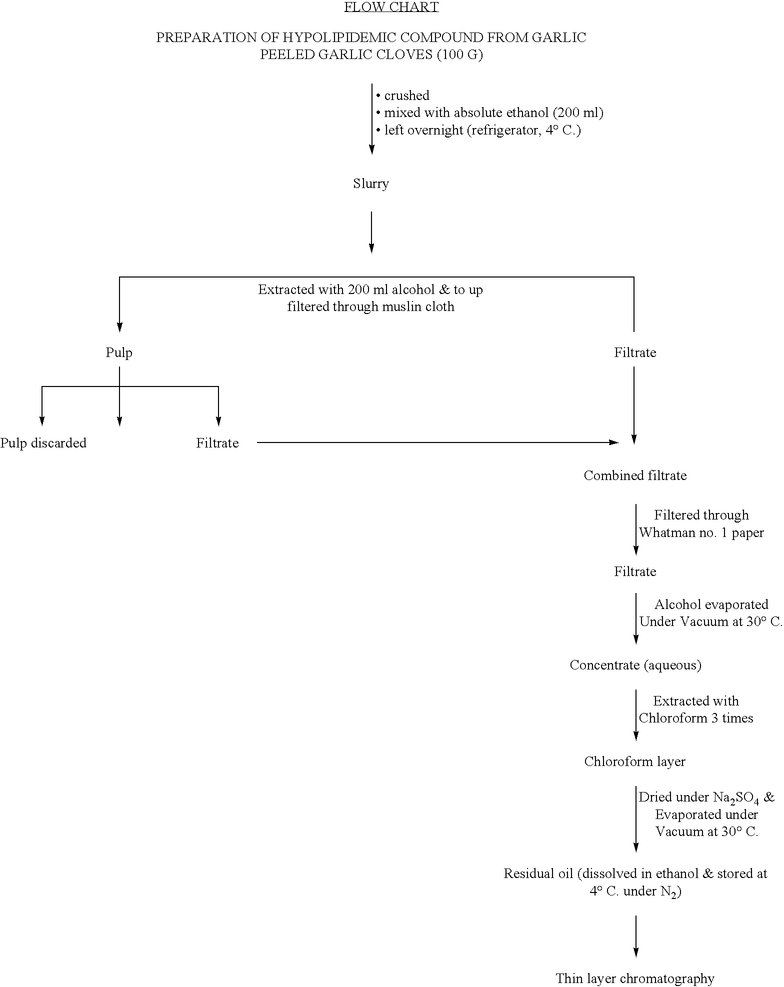

### Example 4

### Product Specifications

Chemical and Physical Properties:

It is a light yellow oil with slight smell of garlic

Biological Properties:

Normally the activity should be tested in hypercholesterolemic rabbits. However, fall in serum cholesterol and triglycerides levels were seen only after treatment with the purified garlic product for six months.

Our studies in Table 2 have shown that the purified garlic product when given to normal rabbits inhibits the incorporation of ¹⁴C acetate into total lipids by 50%. So this has to be used as a measure of biological activity.

Two normal rabbits (about 1 kg.) are given once a day 50 mg/kg. day of the purified garlic products in 0.5 ml of groundnut oil for one week. The liver is dissected, rinsed in chilled saline and tissue was cut into thin slices (approx. 0.5 mm thickness). Slices equivalent 250 mg were taken after removing excess saline by placing them on filter paper and were introduced into 50 ml flasks containing 10 ml phosphate buffer medium. The flasks were closed tightly. The buffer contained the following constituents in mmols/L: 122 NaCl; 1.2 MgSO₄; 1.3 CaCl₂; 0.4 KH₂ PO₄: 17.5 NaHPO₄ and 10 m moles/L of glucose. pH adjusted to 7.8 and 10 μCi of ¹⁴C acetate. Air inside the flask was flushed out with oxygen. The flask was tightly closed incubated in a shaking water bath at 37° C. for one hour. Then the slices were rinsed with large amounts of saline several times to remove free radioactive material. The liver slices were gently blotted with filter paper to remove excess saline and total lipids were isolated from these slices. The tissue was homogenized with about 17 volumes (V/W) of chloroform methanol (2:1 v/v). The fine suspension was then filtered and the homogenizer and funnel were washed with another 2 volumes of chloroform:methanol. The filtrate obtained was shaken well with 0.2 volumes of water in a glass stoppered test tube. The tubes were centrifuged to make the separation of phases complete and to avoid contamination by microdroplets. The upper aqueous phase was removed carefully by a Pasteur pipette. The lower phase was washed with “Folch upper phase” consisting chloroform-methanol water (0.74% KCl) 3:48:47, thrice. The organic phase was evaporated in rotary vacuum evaporator. To this lipid extract, approximately 1-2 ml of benzene was added, mixed and the solvent was evaporated under vacuum. The traces of moisture were removed by drying under a stream of nitrogen. The extract was dissolved in a known volume of chloroform and stored in an air tight container at 20° C. till further analysis. Five normal rabbits treated with only ground nut oil in a similar way served as controls. The purified garlic product shows 50% inhibition of the ¹⁴C the acetate incorporation into total lipids, when compared to that in normal rabbits.

### Example 5

### Effect of the Hypolipidemic Compound from Garlic

Effect of Feeding Purified Garlic Product for Six months on Serum Lipids

As in the previous experiment, three groups of animals (5 each) were taken and group II and III animals received 100 mg/kg/day cholesterol for 6 months. Group III animals received 50 mg/kg/day purified garlic product along with cholesterol for 6 months, while group I animals served as normal healthy controls. At the end of the experiment fasting blood samples were collected for the estimation of various lipid parameters and then animals were sacrificed. Liver, heart and aorta were collected in dry ice and stored at −20° C. until use.

The change in serum lipids are shown in Tables 1 and 2. The mean total serum cholesterol levels increased nearly seven times from 54.8±19.5 mg/dL to 363.4±130.8 mg/dL in group II animals. But the increase in the serum cholesterol levels of purified garlic product treated animals was only 4 times. In this group they rose from 53.5±18.3 to 222.6±139.2 mg/dL. In normal controls these levels showed negligible increase from 49.7±9.7 to 60.8±12.1 mg/dL. Though the difference between the mean values of Groups II and III was notable, it was not statistically significant. This may be due to high standard deviation because of wide difference in individual values.

TABLE 1
Effect of feeding purified garlic product for six months on serum lipid profile
Rabbits of group I did not receive cholesterol and served as normal controls. Group II
animals received cholesterol 100 mg/kg/day, while those in group III received both
cholesterol (100 mg/kg/day) and garlic compound (50 mg/kg/day).

| | Group I | | Group II | | Group III Choles- | | |
|---|---|---|---|---|---|---|---|
| Lipid | Normal | | Cholesterol | | terol + Garlic | | |
| Parameter | Initial | Final | Initial | Final | Initial | Final | ‘p’ Value |
| Total Cholesterol | 49.7 ± 9.7 | 60.8 ± | 54.8 ± | 363.4 ± | 53.5 ± | 222.6 ± | I Vs. II b |
| (mg/dL) | | 12.1 | 19.5 | 130.8 | 18.3 | 139.2 | I Vs III c |
| | | | | | | | II Vs III d |
| HDL-C (mg/dL) | 32.3 ± 5.7 | 36.3 ± 6.5 | 32.2 ± | 41.7 ± | 32.4 ± | 60.7 ± 3.9 | I Vs. II d |
| | | | 13.9 | 17.9 | 13.9 | | I Vs III a |
| | | | | | | | II Vs III c |
| (LDL + VLDL) − C | 17.42 ± 5.5 | 24.5 ± 9.2 | 22.2 ± | 322.9 ± | 21.3 ± | 159.9 ± | I Vs. II a |
| (mg/dL) | | | 15.8 | 121.0 | 4.2 | 139.9 | I Vs III d |
| | | | | | | | I Vs III d |
| (LDL + VLDL) − | 0.53 ± 0.14 | 0.68 ± | 0.86 ± 0.5 | 8.6 ± | 0.67 ± | 5.8 ± 3.9 | I Vs. II b |
| C/HDLC | | 0.24 | | 3.7 | 0.17 | | I Vs III b II |
| | | | | | | | Vs III d |
| T-C/HDL-C | 1.5 ± 0.12 | 1.68 ± | 1.79 ± 0.5 | 9.63 ± | 1.68 ± | 5.4 ± 4.2 | I Vs II b |
| | | 0.24 | | 3.7 | 0.17 | | I Vs III d |
| | | | | | | | II Vs III d |

All the values are mean ± S.D. of 5 animals. Significance is calculated between final values. ‘P’ value ‘a’ < 0.001, ‘b’ < 0.01, ‘c’ < 0.05 and d > 0.05.

In the HDL-cholesterol levels, there was a negligible increase in group TI animals from 32.2±5.7 to 36.3±6.5 mg/dL. Interestingly, in garlic treated animals there was considerable increase in HDL cholesterol from 32.4±13.9 to 60.7±3.9 mg/dL. The increase in these animals was significant compared to untreated hypercholesterolemic animals (P<0.05) and normal controls (P<0.001). About (LDL+VLDL)—cholesterol levels, elevation in group II animals due to cholesterol feeding was very high (nearly 15 fold) from 22.2±15.8 to 322.9±121.0 mg/dL. But in garlic treated animals (group III), the cholesterol induced elevation was only about 7 fold from 21.3±4.2 to 159.9±139.9 mg/dL. The difference between groups II and III (Table 3.7, FIGS. 3.12 & 3.13) is of interest. This means that following garlic administration for 6 months, there was elevation of HDL-cholesterol and decreased elevation of LDL+VLDL both of which are favourable signs. Similar change is seen in the ratio of total cholesterol/HDL-C and (VLDL+LDL)-C/HDL-C. The ratio between total cholesterol and HDL-C was 9.6±3.7 in group II and only 5.4±4.2 in group III compared to 1.68±0.24 of healthy controls (Table 1). Likewise the ratio (LDL+VLDL) C and HDL-C increased to a high value of 8.6±3.7 in group II but only to 5.8±3.9 in group III compared to 0.68±0.24 in normal controls. Thus the increase was less in garlic treated animals (Table 1).

The serum triglyceride levels (Table 2) showed increase to 106.4±91.0 mg/dL in group II but only to 61.5±37.5 mg/dL in group III garlic treated animals. The final mean value of group III rabbits was close to the final value of 78.9±21.2 mg/dL in normal rabbits. This means that serum triglyceride levels were brought down to nearly normal level.

TABLE 2
Effect of feeding purified garlic product for six mouths on serum lipid profile
Rabbits of group I did not receive cholesterol and served as normal controls. Group II
animals received cholesterol 100 mg/kg/day, while those in group III received both
cholesterol (100 mg/kg/day) and garlic compound (50 mg/kg/day).

| | Group I | | Group II | | Group III Choles- | | |
|---|---|---|---|---|---|---|---|
| Lipid | Normal | | Cholesterol | | terol + Garlic | | |
| Parameter | Initial | Final | Initial | Final | Initial | Final | ‘p’ Value |
| Triglycerides | 69.6 ± 19.2 | 78.9 ± 21.2 | 56.7 ± | 106.4 ± | 39.2 ± | 61.5 ± | I Vs II d |
| (mg/dL) | | | 30.3 | 91.0 | 6.4 | 37.5 | I Vs III d |
| | | | | | | | II Vs III d |
| Phospholipids (mg | 3.2 ± 0.65 | 3.4 ± 0.6 | 3.36 ± | 7.3 ± | 3.0 ± | 6.27 ± | I Vs II c |
| phospholipid | | | 0.5 | 2.4 | 0.4 | 2.4 | I Vs III c |
| phosphorus/dL) | | | | | | | II Vs III d |
| Free fatty acids | 420.0 ± | 454.0 ± | 435.0 ± | 433.0 ± | 442.5 ± | 490.0 ± | I Vs II d |
| (μmols/L) | 68.9 | 84.1 | 166.0 | 81.4 | 94.6 | 57.7 | I Vs III d |
| | | | | | | | II Vs III d |

All the values are mean ± S.D. of 5 animals. Significance is calculated between final values. ‘P’ value ‘a’ < 0.001, ‘b’ < 0.01, ‘c’ < 0.05 and d > 0.05.

The serum phospholipid levels went up to 7.3±2.4 mg phospholipid phosphorus (PLP)/dL in the untreated hypercholesterolemic (group II) rabbits and to 6.27±2.4 mg PLP/dL in garlic treated rabbits compared to 3.4±0.6 mg PLP/dL in normal controls (Table 2). It is known that the serum phospholipids correlate positively with serum cholesterol. The slight fall in phospholipids after treatment agrees with serum cholesterol.

The levels of plasma free fatty acids (FFA) did not show any increase on feeding either with cholesterol or cholesterol plus garlic. The plasma FFA content was equal to that of normal controls (Table 2).

On the whole if percentage changes are taken into account serum total cholesterol, triglycerides, HDL-cholesterol and (LDL+VLDL)-cholesterol levels in garlic treated groups of rabbits showed considerable improvement. However, on statistical evaluation the difference between the untreated and treated groups was not significant in some of the parameters (Cholesterol) due to wide variations in the values between the individual animals. These variations are more prominent in the animals challenged with dietary cholesterol. The responsiveness of individual animals seems to play very important role in this phenomenon, which is a genetic character. On the whole the results in the present study of the effect of purified garlic product on serum lipids are in agreement with some reports of previous workers. Augusti and Mathew 1974, fed normal rats with allicin for 2 months, Qureshi et al. (1983 a, b) fed normal chicken with garlic extracts for one month and observed similar changes.

Effect of Feeding Purified Garlic Product for Six Months on Tissue Lipids

Liver, heart and aorta of the animals were analysed for their lipid content. The results are summarised in Tables 3, 4 and 5.

As seen in the Table 3 the total lipids in the liver of untreated hypercholesterolemic (group II) animals increased to 41.2±6.1 mg/g, compared to 30.5±5.1 mg/gm tissue in normal group (group I) of animals. The increase was highly significant (P<0.001). The total lipid content in cholesterol plus garlic treated animals (group III) did not increase at all and was equal to that of normal animals (29.8±8.8 mg/g). The difference between groups II and III was statistically significant (P<0.05). This shows that with our garlic compound treatment there was total prevention of cholesterol induced lipid accumulation in liver which occurred in untreated animals. It can not be stated whether this was due to inhibition of the synthesis or increased mobilization of tissue lipids. This aspect was studied and mentioned later.

TABLE 3
Effect of feeding purified garlic product for six months on the
composition of lipids in the liver
Rabbits of group I did not receive cholesterol and served as normal
controls. Group II animals received cholesterol 100 mg/kg/day, while
those in group III received both cholesterol (100 mg/kg/day) and
purified garlic product (50 mg/kg/day).

| | | | Group III | |
|---|---|---|---|---|
| Lipid | Group I | Group II | Choleste- | ‘p’ |
| Parameter | Normal | Cholesterol | rol + Garlic | Value |
| Total lipids | 30.5 ± 5.1 | 41.2 ± 6.1 | 29.8 ± 8.8 | I Vs II a |
| (mg/g tissue) | | | | I Vs III d |
| | | | | II Vs III c |
| Total Cholesterol | 2.4 ± 0.4 | 11.0 ± 2.0 | 8.0 ± 1.8 | I Vs II a |
| (mg/g tissue) | | | | I Vs III a |
| | | | | II Vs III c |
| Free Cholesterol | 1.8 ± 0.2 | 3.8 ± 0.6 | 3.3 ± 0.5 | I Vs II a |
| (mg/g tissue) | | | | I Vs III b |
| | | | | II Vs III d |
| Cholesterol esters | 0.3 ± 0.1 | 6.0 ± 1.5 | 4.3 ± 1.0 | I Vs II a |
| (mg/g tissue) | | | | I Vs III a |
| | | | | II Vs III d |
| Triglycerides | 2.3 ± 0.4 | 4.8 ± 1.8 | 1.8 ± 0.8 | I Vs II c |
| (mg/g tissue) | | | | I Vs III d |
| | | | | II Vs III c |
| Phospholipids | 1.6 ± 0.3 | 1.27 ± 0.19 | 1.1 ± 0.1 | I Vs II c |
| (mg lipid | | | | I Vs III c |
| Phosphorus/ | | | | II Vs III d |
| g (tissue) | | | | |

All the values are mean ± S.D. of 5 animals. Significance is calculated between final values. ‘p’ value ‘a’ < 0.001, ‘b’ < 0.01, ‘c’ < 0.05 and ‘d’ > 0.05.

Since garlic treatment showed considerable decrease in the total lipids of liver, it was intended to see whether the favourable change was in all or only few of the individual lipid fractions. When individual lipids were estimated, it was found that the total cholesterol content in liver was elevated in group II animals (11.0±2.0 mg/g) compared to normal controls (group I) 2.4±0.4 mg/g (P<0.001). In garlic fed group the increase in the total cholesterol content was much less (8.0±1.8 mg/g) compared to that in the untreated group. The difference was of borderline significance (P=0.05). The increase was relatively more in cholesterol esters than free cholesterol. Free cholesterol levels were found to be 3.8±0.6 mg/g in group II animals and 3.3±0.5 mg/g in those of group III, compared to 1.8±0.2 mg/g in normal control Group I of rabbits. However, there was no significant difference between the untreated and treated animals as seen in Table 3. The increase in cholesterol ester content was more in untreated animals but less in garlic treated hypercholesterolemic animals (P<0.001) compared to normal animals (0.3±0.1 mg/g). This value was elevated to 6.0±1.5 and 4.3±1.0 mg/g) in groups II and III respectively. Thus our purified garlic product prevented the accumulation of cholesterol esters and to some extent free cholesterol in liver. The lower level of total cholesterol in the liver of garlic treated animals could be due to its conversion to bile acids as stated by Chi et al (1982).

Purified garlic product feeding had remarkable effect on triglyceride content in liver. In untreated hypercholesterolemic animals its levels went up to 4.8±1.8 mg/g compared to 2.3±0.4 mg/g in healthy controls (P<0.05). But interestingly the amount in garlic treated animals (1.8±0.8 mg/g) was lower than the levels found in normal control animals (Table 3). It means that garlic product could completely prevent the accumulation of triglycerides inspite of cholesterol feeding. One of the possible explanations is the inhibition of synthesis of triglycerides by garlic. The data on the incorporation of 14C-acetate into triglycerides (shown later) in garlic treated rabbits support this view. An important observation of Chi et al (1982), that garlic feeding inhibited glucose-6 phosphate dehydrogenase and malic enzyme which play an important role in lipid synthesis, also supports the above view.

Phospholipid content is the only lipid component found decreased in untreated and treated animals. Phospholipid content was 1.27±0.19 and 1.1±0.1 mg PLP/g respectively in groups II and III compared to 1.6±0.3 in normal animals (Table 3). Augusti and Mathew (1974) also observed decreased phospholipid content in the liver of allicin fed normal rats.

Another interesting observation of high significance has been total prevention of triglyceride and partial reduction of cholesterol deposition in aorta. The total cholesterol content (Table 4) increased to 3.6±1.2 mg/g in untreated hypercholesterolemic animals and to only 2.0±0.35 mg/g in our garlic product treated hypercholesterolemic rabbits compared to 0.9±0.18 mg/g in normal animals. The difference between groups II and III values was also significant P<0.05. The triglyceride content of the aorta of garlic treated (22.3±6.6 mg/g) rabbits was equal to that of normals (21.4±3.3 mg/g) when compared with a high value of 35.2±10.3 mg/g tissue in cholesterol fed animals. This is a very encouraging result since the development of fatty streak which later transforms into atherosclerotic plaque begins by accumulation of cholesterol and triglycerides in the arterial wall.

TABLE 4
Effect of feeding purified garlic product for six months on the
composition of lipids in the aorta
Rabbits of group I did not receive cholesterol and served as normal
controls. Group II animals received cholesterol 100 mg/kg/day, while
those in group III received both cholesterol (100 mg/kg/day) and purified
garlic product (50 mg/kg/day).

| | | | Group III | |
|---|---|---|---|---|
| Lipid | Group I | Group II | Choleste- | ‘p’ |
| Parameter | Normal | Cholesterol | rol + Garlic | Value |
| Total Cholesterol | 0.9 ± 0.18 | 3.6 ± 1.25 | 2.0 ± 0.35 | I Vs II b |
| (mg/g tissue) | | | | I Vs III a |
| | | | | II Vs III c |
| Triglycerides | 21.4 ± 3.3 | 35.2 ± 10.3 | 22.3 ± 6.6 | I Vs II c |
| (mg/g tissue) | | | | I Vs III d |
| | | | | II Vs III d |

All the values are mean ± S.D. of 5 animals. Significance is calculated between final values. ‘p’ value ‘a’ < 0.001, ‘b’ <0.01, ‘c’ < 0.05 and ‘d’ > 0.05.

Though there was no significant change in the lipids of heart after 2 months feeding of garlic product, longer treatment for 6 months showed some interesting changes. Total lipids increased (Table 5) in untreated hypercholesterolemic rabbits significantly (35.7±3.0 mg/g), but their increase was prevented almost completely in garlic product fed animals (27.4±3.2 mg/g). Even the individual lipids, cholesterol and triglycerides of the heart showed similar behaviour. The change in the case of triglycerides is more remarkable. Thus the major effect of garlic product in cholesterol fed animals seems to be to prevent the accumulation and normalize the levels of triglycerides in serum, liver, aorta and heart.

TABLE 5
Effect of feeding purified garlic product for six months on the
composition of lipids in the heart
Rabbits of group I did not receive cholesterol and served as normal
controls. Group II animals received cholesterol 100 mg/kg/day, while
those in group III received both cholesterol (100 mg/kg/day) and
(50 mg/kg/day).

| | | | Group III | |
|---|---|---|---|---|
| Lipid | Group I | Group II | Cholesterol | ‘p’ |
| Parameter | Normal | Cholesterol | Garlic | Value |
| Total lipids mg/g | 23.04 ± 4.6 | 35.7 ± 3.0 | 27.4 ± 3.2 | I Vs II a |
| | | | | I Vs III d |
| | | | | II Vs III b |
| Total Cholesterol | 1.3 ± 0.16 | 1.66 ± 0.36 | 1.5 ± 2 ± | I Vs II d |
| mg/g | | | 0.04 | I Vs III c |
| | | | | II Vs III d |
| Triglycerides | 4.26 ± 1.1 | 5.56 ± 1.8 | 3.8 ± 0.66 | I Vs II d |
| | | | | I Vs III d |
| | | | | II Vs III d |

All the values are mean ± S.D. of 5 animals. Significance is calculated between final values. ‘p’ value ‘a’ < 0.001, ‘b’ <0.01, ‘c’ <0.05 and ‘d’ >0.05.

### Example 6

### Mechanism of Action of Purified Garlic Hypolipidemic Compound

Experimental evidence available at present suggests mainly three possible mechanisms:

(a) Inhibition or reduction in lipogenesis.

(b) Mobilization of lipids from tissues into circulation and ultimate excretion.

(c) Increased catabolism of lipids and enhanced elimination of metabolic by-products.

These results are believed to be achieved by altering the activity of many enzymes involved in lipid synthesis. Augusti & Mathew (1974) has attributed the lipid lowering effect to the ability of garlic principles to block-SH groups especially of coenzyme A which is essential for the biosynthesis of fatty acids, cholesterol, triglycerides and phospholipids. Garlic feeding also inhibited the lipogenic enzymes glucose-6-phosphate dehydrogenase and malic enzyme (Chi et al 1982). Qureshi et al (1983 a, b) have demonstrated the inhibition of important lipid synthesizing enzymes like HMG CoA reductase and fatty acid synthetase by extracts of garlic. The incorporation of 14 C-acetate into lipids is inhibited by garlic (Chang & Johnson 1980, Kritchevsky et al 1980). More recently it has been shown that garlic inhibits the acetyl CoA synthetase in yeast and mammalian tissues (Focke et al 1990). Inhibition of many other enzymes by garlic and its products is also reported by Sodmu et. al (1984), Adoga and Osuoi (1986). We, therefore, studied the effect of the purified garlic product on some enzymes.

### Example 7

### Effect on Cholesterol Biosynthesis in Liver

The effect of our garlic product feeding on cholesterol biosynthesis was studied by calculating the ratio between HMG CoA and mevalonate estimated by the method of Rao et al. ( ). We are fully aware that this method gives only the indirect assessment of HMG CoA reductase activity which is a rate limiting step in the biosynthesis of cholesterol. The direct and most desirable method of using the 14C-HMG CoA could not be followed because it is very expensive.

The ratio (Table 6) in untreated hypercholesterolemic animals (11.37±1.78) as compared to normal animals (8.97)±2.9), indicates the inhibition of cholesterol biosynthesis due to cholesterol feeding to these animals. But interestingly the ratio decreased in purified garlic product treated animals to a value (7.44±1.27) lower to that even in normal animals. This suggests that inhibition of cholesterol biosynthesis by exogenous cholesterol has been removed by garlic product treatment probably by increasing the conversion of cholesterol to other products like bile acids as indicated by other workers. The difference in the ratio between groups II and III was significant (P<0.01). The reduction of lipids and suppression in the levels of cholesterol in particular, by garlic is a well known fact. But the exact mechanism was not proved conclusively till recently. Qureshi and coworkers (1983 a,b) reported that water and organic solvent extracts of garlic inhibited the that hepatic HMG CoA reductase, the regulatory enzyme in cholesterol biosynthesis. Kumar et al (1991) also reported in vitro inhibition of HMG CoA reductase activity by dially disulphide obtained from garlic, which was proposed to be the result of formation of protein disulfides inaccessible for reduction by thiol agents.

Effect of Purified Garlic product feeding for 6 months on Liver HMG
COA reductase activity (expressed as ratio between HMG
CoA/Mevalonate)
Rabbits of group I did not receive cholesterol and served as normal
controls. Group II animals received cholesterol 100 mg/kg/day, while
those in group III received both cholesterol (100 mg/kg/day) and purified
garlic product (50 mg/kg/day).

| Group | HMG CoA/mevalonate | ‘p’ value |
|---|---|---|
| I Normal | 8.97 ± 2.9 | |
| II Cholesterol | 11.37 ± 1.78 | I Vs II d |
| | | I Vs III d |
| | | II Vs III b |
| III Cholesterol + garlic product | 7.44 ± 127 | |

All the values are mean ± S.D. of 5 animals. Significance is calculated between final values. ‘p’ value ‘a’ < 0.001, ‘b’ < 0.01 ‘c’ < 0.05 and ‘d’ > 0.05.

### Example 8

### Incorporation of ¹⁴C-Acetate into Lipids in Liver Slices In Vitro.

Incorporation of ¹⁴C-acetate into lipid fractions in the liver slices of treated and untreated hypercholesterolemic animals was studied. The results are summarised in Table 7. Incorporation of ¹⁴C-acetate into total lipids increased (7687±1289 cpm) significantly (p<0.05) in cholesterol fed animals (group II) when compared to normal control (group I) (5320±626 cpm).

TABLE 7
Incorporation of ¹⁴C-Acetate into the lipids of liver in vitro
Rabbits of group I did not receive cholesterol and served as normal
controls. Group II animals received cholesterol 100 mg/kg/day, while
those in group III received both cholesterol (100 mg/kg/day) and purified
garlic product (50 mg/kg/day).

| | Radio Activity (cpm/gm tissue) | | | |
|---|---|---|---|---|
| | | | Group III | |
| | Group I | Group II | Choleste- | |
| Lipid Fractions | Normal | Cholesterol | rol + Garlic | ‘P’ Values |
| Total lipids | 5320 ± 626 | 7687 ± 1289 | 3898 ± 867 | I Vs II c |
| | | | | I Vs III c |
| | | | | II Vs III b |
| Free Cholesterol | 874 ± 588 | 488 ± 174 | 511 ± 160 | I Vs II d |
| | | | | I Vs III d |
| | | | | II Vs III d |
| Cholesterol esters | 458 ± 179 | 458 ± 252 | 327 ± 115 | I Vs II d |
| | | | | I Vs III d |
| | | | | II Vs III d |
| Triglycerides | 1049 ± 87 | 1424 ± 330 | 1023 ± 412 | I Vs II c |
| | | | | I Vs II d |
| | | | | I Vs III d |
| Phospholipids | 2475 ± 514 | 2380 ± 336 | 1838 ± 525 | I Vs II d |
| | | | | I Vs III d |
| | | | | II Vs III d |

All the values are mean ± S.D. of 5 animals. Significance is calculated between final values. ‘p’ value ‘a’ < 0.001, ‘b’ < 0.01, ‘c’ < 0.05 and ‘d’ > 0.05

But in garlic treated animals (group III) the incorporation decreased to a value (3898±867 cpm) (P<0.05) less than that in normal animals. The inhibition observed was statistically significant (P<0.01) between groups II and III. In order to see into which fraction of lipids the incorporation of ¹⁴C-acetate took place, the individual lipids were separated by thin layer chromatography into free and esterified cholesterol, triglycerides and phospholipids and radioactivity was counted (Table 7).

The increase in incorporation (40%) of ¹⁴C-acetate into total lipids seen in cholesterol treated group was accounted for mostly by the increase in triglycerides (37%) as can seen from Table 7. Interestingly garlic product could prevent not only the increase but also brought down the incorporation to a value less than that of healthy controls in some animals. This implies that the action of our garlic product in this particular instance was by inhibition of ¹⁴C acetate incorporation into triglycerides. There was not much change in the incorporation of ¹⁴C-acetate into cholesterol esters and phospholipids in cholesterol fed animals. However in the garlic product treated group there was slight inhibition of ¹⁴C-acetate incorporation (25-30%) into cholesterol esters when compared with healthy controls, which however was not statistically significant (P<0.05). Since cholesterol was given to rabbits of group II and III, there was decreased incorporation of ¹⁴C acetate into cholesterol, which is probably because exogenous cholesterol has inhibitory effect (perhaps feed back). Obviously the effect of garlic product on cholesterol biosynthesis can not be studied while giving cholesterol to the animals along with garlic as in this experiment. It becomes necessary to study the effect of garlic product in normal animals. But this experiment and the data on tissue and serum triglycerides levels prove that the purified garlic product can prevent the synthesis of triglycerides from acetate in cholesterol induced hypertriglyceridemia and thereby prevent the accumulation of triglycerides in liver and in serum.

Feeding garlic product inhibited the incorporation of ¹⁴C acetate into phospholipids also.

### Example 9

### Effect of Purified Garlic Product Feeding for Six Months on Carbohydrate Metabolism

Effect on Fasting Blood Glucose (FBG)

There was no significant difference between the normal group of animals or cholesterol or cholesterol plus the purified garlic product treated groups either in the fasting blood glucose values or on glucose tolerance (results not shown). On the contrary, many workers have reported hypoglycemic activity of garlic (Brahmachari and Augusti 1962 Jain and Vyas 1975). The reason could be that we studied in hypercholesterolemic rabbits but others involved diabetic animals. Chang and Johnson (1980) have demonstrated in rate that feeding of garlic lowered serum glucose levels by raising serum insulin levels.

Effect of Garlic Product on Glucose Metabolism

The liver glycogen content significantly (P<0.001) increased in garlic product fed rabbits when compared with normal and untreated cholesterol fed animals. Chang and Johnson (1980) also observed increase in glycogen content in garlic fed normal rats.

We also studied the incorporation of ¹⁴C-glucose into liver glycogen. Liver slices from purified garlic product fed animals showed significant increase in radioactivity in glycogen when compared to normal and untreated cholesterol fed animal groups (results not shown). The reason for increased ¹⁴C-glucose incorporation into glycogen of untreated cholesterol fed than normal rabbits is not known. Chang and Johnson (1980) observed increased radioactivity from ¹⁴C-sucrose into liver glycogen in rats fed garlic supplemented diet. They attributed this to increase in serum insulin which also promotes the conversion of inactive glycogen synthetase to active form thus leading to increased glycogen synthesis. Purified garlic product not only increased the incorporation of ¹⁴C-glucose into glycogen but also increased glycogen content. It is to be presumed that purified garlic product improved glycogen synthesis by increasing serum insulin levels.

Effect of Garlic Product on Glycolysis and Gluconeogenesis

Effect of feeding garlic product for six months on one key enzyme each in glycolysis and gluconeogenesis pathways was studied.

There was a significant decrease in the activity of glucose-6-phosphatase in the garlic treated and untreated hypercholesterolemic animals when compared to healthy animals. The difference in the activity between groups II and III rabbits was not significant (Table 8). This shows that the decrease in the activity was due to cholesterol feeding but not due to allicin.

Similar results were observed in the activity of glucokinase. There was a significant increase in the activity of this enzyme in untreated and garlic product treated hypercholesterolemic animals compared to healthy animals, and the difference between these two cholesterol fed groups was not statistically significant (Table 3.14). This again shows that cholesterol feeding increases the activity of glucokinase, a key enzyme in glycolysis pathway.

On the whole cholesterol feeding itself inhibited one key enzyme in gluconeogenesis and activated a key enzyme in glycolysis pathway. Consequently, it is difficult to draw any conclusion regarding the effect of our garlic product on glucokinase and glucose-6-phosphatase on normal animals.

Effect of Purified Garlic Product Feeding to Normal Rabbits for One Week.

As mentioned above, the main emphasis of our studies has been to see the purified garlic product whether or not produces favourable changes in hypercholesterolemic animals. However it is also necessary to find out whether it has favourable effect on normal animals since garlic is used in diet. Further, this study would enable comparison of the effect of the garlic product on hypercholesterolemic and normal rabbits

TABLE 8
Effect of purified garlic product feeding for six months on liver
glucose-6-phosphatase and glucokinase activity.
Rabbits of group I did not receive cholesterol and served as normal
controls. Group II animals received cholesterol 100 mg/kg/day, while
those in group III received both cholesterol (100 mg/kg/day) and garlic
product (50 mg/kg/day).

| | Glucose-6-phosphatase | |
|---|---|---|
| | (μmoles phosphate | Glucokinase |
| Group | liberated/min/g tissue) | (Units/mg. Protein) |
| I Normal | 12.42 ± 0.98 | 0.096 ± 0.04 |
| II Cholesterol | 5.51 ± 0.71 | 0.24 ± 0.05 |
| III Cholesterol | 6.1 ± 1.49 | 0.218 ± 0.04 |
| garlic product | I vs II a | I vs II b |
| | I vs III a | I vs III b |
| | II vs III d | II vs III d |

Al the values are mean ± S.D. of 5 animals. Significance is calculated between final values. ‘p’ values ‘a’ < 0.001, ‘b’ < 0.01, ‘c’ < 0.05 and ‘d’ > 0.05

Two groups of animals (5 each) were taken. Their serum total cholesterol and triglycerides were estimated to give initial values in both the groups. Animals in group I were kept untreated and served as healthy controls, whereas group II animals were given 50 mg/kg/day purified garlic product (along with 0.5 ml groundnut oil) orally for one week. Animals of group I received the same volume of oil. At the end of one week, blood samples were collected from overnight fasted animals for serum lipid profile. The animals were sacrificed, their livers removed and slices were incubated with ¹⁴C-acetate to study incorporation into lipids as described earlier in methods.

Purified garlic product when fed to the normal rabbits along with small amount (0.5 ml) of oil for one week, showed surprising results (Table 9). Serum total cholesterol levels increased from 59.5+10.4 mg/dl to 205.9±40.7 mg/dL in group II compared to 53.0±13.2 from 52.7±18.2 mg/dL in control group. The difference between the final values was statistically significant (p<0.01). In the same way, serum triglycerides also increased to 255.4±102.2 mg/dL from an initial value of 57.7±20.4 mg/dL in group II, while in control animals the values remained within normal range (58.3+10.5 to 64.6±11.6 mg/dL). The difference between the final values was statistically significant (p<0.05). This represents an increase of 288% in serum cholesterol and 295% in triglycerides. In all probability the increase in the serum lipids seen in normal animals after garlic product could also be due to mobilization of tissue lipids observed in the case of animals fed garlic product and cholesterol simultaneously for 6 months (Table 9).

TABLE 9
Effect of purified garlic product feeding for one week on serum lipids of
normal rabbits
Rabbits of group I received normal diet and served as healthy controls.
Group II animals also received normal diet but were given garlic product
(50 mg/kg/day) orally.

| | Group I | | Group II | | |
|---|---|---|---|---|---|
| Serum Lipid | Normal | | treated | | ‘p’ |
| parameter | Initial | Final | Initial | Final | Value |
| Total- | 52.7 ± 18.2 | 53.0 ± 13.2 | 59.5 ± 10.4 | 295.9 ± | b |
| cholesterol | | | | 40.7 | |
| (mg/dL) | | | | | |
| Triglycerides | 58.3 ± 10.5 | 64.6 ± 11.6 | 57.7 ± 20.4 | 255.4 ± | c |
| (mg/dL) | | | | 102.2 | |

All the values are mean ± S.D. of 5 animals. Significance is calculated between final values. ‘p’ value ‘a’ < 0.001, ‘b’ < 0.01, ‘c’ < 0.05 and d > 0.05.

The lowering of the ratio of HMG CoA/mevalonate (Table 10) could be explained on the basis of the ability of the purified garlic product to inhibit the formation of acetyl CoA. This is supported by our results in Table 11, from which it can be seen that the garlic product feeding decreased the incorporation of ¹⁴C-acetate into total lipids (53%), free cholesterol (30%), esterified cholesterol (70%), triglycerides (51%) and phospholipids (39%). Nearly 50-70% reduction in the incorporation of ¹⁴C-acetate into cholesterol esters, triglycerides and total lipids is indicative of the favourable effects of purified garlic product in a short time (one week) to normal animals.

Effect of Purified Garlic Product on Liver Functions.

If garlic product is to be given for a long period, it is necessary to know whether it has any toxic effects on long term administration. For this purpose, in the rabbits treated with the purified garlic product and cholesterol for six months, some blood parameters of liver functions and histopathological changes in aorta, heart and liver were investigated (Table 12). Since garlic product (50 mg/mg/day) was given along with cholesterol (100 mg/kg/day) for six months, untreated cholesterol fed animals but not healthy controls, were used for comparison.

The serum protein levels of normal (4.9±0.2 g/dL) and untreated hypercholesterolemic (4.8±0.4 g/dL) purified garlic product and treated hypercholesterolemic animals (5.2±0.1 g/dL were more or less equal.

TABLE 10
Effect of purified garlic product feeding to normal rabbits for one week
on HMG CoA reductase activity in liver
Rabbits of group I received normal diet and served as healthy controls.
Group II animals also received normal diet but were given purified garlic
product (50 mg/kg/day) orally.

| | Group I | Group II | |
|---|---|---|---|
| | Normal | Garlic Treated | ‘p’ value |
| HMG CoA/mevalonate | 8.97 ± 2.9 | 1.28 ± 0.02 | I vs II a |

The ratio between HMG CoA to mevalonate was taken as an index of enzyme activity. All the values are mean ± SD of 5 animals. ‘P’ value ‘a’ < 0.001; b < 0.01; c < 0.05 and d > 0.05

TABLE 11
Effect of purified garlic product feeding to normal rabbits for one week on
¹⁴⁻C acetate incorporation into lipids.
Rabbits of group I received normal diet and served as healthy controls.
Group II animals also received normal diet but were given purified garlic
product (50 mg/kg/day) orally.

Radio activity (cpm/g tissue)

| | Group I | Group II | |
|---|---|---|---|
| Lipid fraction | Normal | Garlic treated | % inhibition |
| Total lipids | 5320 ± 626 | 2470 ± 587 | 53 |
| Free cholesterol | 874 ± 580 | 612 ± 50 | 30 |
| Cholesterol esters | 458 ± 179 | 138 ± 49 | 70 |
| Triglycerides | 1049 ± 87 | 514 ± 188 | 51 |
| Phospholipids | 2475 ± 514 | 1500 ± 163 | 39 |

All the values are mean ± S.D. of 5 animals.

TABLE 12
Effect of feeding purified garlic product for six months on liver function
tests
Rabbits of group I did not receive cholesterol and served as normal
controls. Group II animals received cholesterol 100 mg/kg/day, while
those in group III received both cholesterol (100 mg/kg/day) and purified
garlic product (50 mg/kg/day).

| | Group I | Group II | Group III | |
|---|---|---|---|---|
| | Normal | Cholesterol | Cholesterol | ‘P’ Values |
| Serum proteins | 4.9 ± 0.2 | 4.8 ± 0.4 | 5.2 ± 0.1 | I Vs II d |
| (g/dL) | | | | II Vs III c |
| | | | | II Vs III d |
| Serum bilirubin | 0.27 ± 0.04 | 0.3 ± 0.07 | 0.32 ± 0.1 | I Vs II d |
| (mg/dL) | | | | I Vs III d |
| | | | | II Vs III d |
| Serum Alkaline | 2.8 ± 0.3 | 3.8 ± 0.6 | 3.9 ± 1.4 | I Vs II c |
| Phosphatase (KA | | | | I Vs III d |
| Units/dL) | | | | II Vs III d |
| Serum ALT | 24.7 ± 6.5 | 24.1 ± 3.9 | 25.2 ± 5.4 | I Vs II d |
| (IU/L) | | | | I Vs III d |
| | | | | II Vs III d |

All the values are mean ± S.D. of 5 animals. Significance is calculated between final values. ‘P’ value ‘a’ < 0.001, ‘b’ < 0.01, ‘c’ < 0.05 and ‘d’ > 0.05.

This shows that the purified garlic product treatment had no effect on serum protein levels. Serum total bilirubin levels remained normal in all the three groups. The values were 0.27±0.04, 0.3±0.07 and 0.32±0.1 mg/dL in normal, cholesterol fed untreated and cholesterol fed garlic product treated animals respectively. Serum alkaline phosphatase values in normal, untreated hypercholesterolemic and garlic product treated hypercholesterolemic animals were 2.8±0.3, 3.8±0.6 and 3.9±1.4 KA units/dL respectively. This indicates that the garlic product treatment had no adverse effect on serum alkaline phosphatase levels.

Serum alanine amino transferase (ALT) or glutamate pyruvate transaminase (SGPT) levels did not change on feeding cholesterol or cholesterol plus garlic product for six months. The ALT levels were 24.7±6.5, 24.1±3.9 and 25.2±5.4 IU/dL in groups I, II and III respectively.

The above liver function tests show that treatment with the purified garlic product for six months did not have any adverse effect on the functions of liver as assayed by the above serum parameters.

### Example 10

### Histopathology

Aorta

Histomorphological changes either associated with or suggestive of treatment could not be seen in both groups II and III. Only one rabbit of (group II) cholesterol fed untreated group had focal hyaline necrosis of tunica media associated with cavitation. However, no evidence of deposition of cholesterol crystals could be seen.

Heart

The predominant histopathological changes observed in cardiac muscle are fatty changes, loss of striation and nuclei and occasional necrosis. Presence of signet-ring appearing nuclei were seen in many cells. However, these changes were observed in both cholesterol fed and garlic product treated groups and therefore not associated with garlic product treatment. In one case from cholesterol fed group there was a distinct area of necrosis and sarcolemmal proliferation.

Liver

Few hepatic lesions were noticed in the animals. Periductal and periportal moderate lymphomononuclear cell infiltration, periportal fibroplasia and mild to moderate biliary hyperplasia, multifocal but tiny mononuclear cell infiltration, microabscesses and mild to moderate fatty changes in hepatic parenchyma are the changes observed in both the groups. The hepatic lesions are therefore, not treatment associated.

From the above mentioned liver function tests and histopathological studies it is clear that administration of the garlic product at the therapeutic dose for six months did not have any adverse effects. However, these are not typical toxicity studies for two reasons:

a) Purified Garlic Product was given along with cholesterol but not alone.

b) Purified garlic product was given only at therapeutic dose but not at a higher dose as required for chronic toxicity studies.

### REFERENCES

The following references were referred to herein:

1. Adoga, G. I. and Osuji, J. (1986): Effect of garlic oil extract on serum, liver and kidney enzymes of rats fed on high sucrose the alcohol diets. Biochem. Int. 13: 615-24.

2. Arora, R. C. and Arora, S. (1981): Comparative effect of clofibrate, garlic oil and onion on alimentary hyperlipemia. Atheresclerosis 39; 447-52.

3. Augusti, K. T. (1977): Hypocholesterolaemic effect of garlic, Allium sativum Linn. Indian. J. Exp. Biol. 15: 489-90.

4. Augusti, K. T. and Mathew, P. T. (1974): Lipid lowering effect of allicin (diallyl disulfide-oxide) on long term feeding to normal rats. Experientia 30: 468-70.

5. Bilheimer, D. W. Grundy, S. M. Brownn M. S. and Goldstein, J. L. (1983): Mevinolin and colestipol stimulate receptor-mediated clearance of low density lipoprotein from plasma in familial hypercholesterolemia heterozygotes. Prec. Natl. Acad Sci; USA. 80: 4124-8.

6. Bordia, A, Arora, S. K, Kothari, L. K., Jain, K. C., Rathore, B. S., Rathore, A. S., Dube, M. K., Bhu, N. (1975): The protective action of essential oils of onion and garlic in cholesterol-fed rabbits. Atherosclerosis 22: 103-9.

7. Bordia, A., Verma, S. K., Byas, A. K., Khabya, B. L., Rathore, A. S., Bhu, N., and Bedi, H. K. (1977a): Effect of essential oil of onion and garlic on experimental atheresclerosis in rabbits. Atherosclerosis 26: 379-86.

8. Bordia, A. (1981): Effect of garlic on blood lipids in patients with coronary heart disease. Am. J. Clin. Nutr. 34: 2100-3.

9. Brahmachari, H. D. and Augusti, K. T. (1962): Orally effective hypoglycemic agents from plants. J. Pharma. Pharmacol. 14: 254.

10. Carlson, L. A. and Osson, A. G. (1979): Effect of drugs on lipoorotein metabolism. Prog. Biochem. Pharmacol. 15: 238.

11. Chang, M. L. W. and Johnson, M. A. (1980): Effect garlic on carbohydrate metabolism and lipid synthesis in rats. J. Nutr. 110: 931.

12. Chi, M S, Koh E T, Stewart T J et. al (1982): Effect of garlic on lipid metabolism in rats fed cholesterol or lard. J. Nutr. 112: 241-8.

13. Hoeg J M, Schaefer E J, Romano C A, Bou E, Pikus A M, Zech L A, Bailey K R, Gregg R E, Wilson P W, Sprecher D L, et al (1984): Neomycin and plasma lipoproteins in type II hyperlipoproteinemia. Clin. Pharmacol. Ther. 36: 555-65.

14. Imada, O. (1993): Toxicity aspects of garlic. First world congress on the health significance of garlic and garlic constituents. Washington, USA.

15. Jain, R. C. (1976): Onion and garlic in experimental cholesterol induced atherosclerosis. Ind. J. Med. Res. 74.1509.

16. Jain, R. C. and Konar, D. B. (1977): Medikon 6, 15.

17. Jain, R. C. and Konar, D. B (1978): Effects of garlic oil on experimental cholesterol atherosclerosis. Atherosclerosis 29: 125-9.

18. Jain, R. C. and Vyas C. R. (1975): Garlic in alloxan induced diabetic rabbits. Am. J. Clin. Nutr. 28: 684.

19. Kamanna V. S. and Chandrasekhara, N (1982): Effect of garlic (Allium sativum Linn.) on serum lipoproteins and lipoprotein cholesterol levels in albino rats rendered hypercholesterolemic by feeding cholesterol. Lipids 17: 483.

20. Kane, J. P; Malloy, M. J; Tun, P; Philips, N. R; Freedman, D. D; Williams, M. L; Rowee, I. S. and Havel, R. J. (1981): Normalization of low density—lipoprotein levels in heterozygous familial hypercholesterolemia with a combined drug regimen. New Engl. J. Med. 304: 251-8.

21. Kane, J. P. and Malloy, M. J. (1982): Treatment of hypercholesterolemia. Med. Clin. North Am. 66: 537-50.

22. Kumar, R. V.; Banerji, A; Kurup, C. K. and Ramasarma, T. (1991): The nature of inhibition 3-hydroxy-3-methylglutaryl CoA reductase by garlic derived diallyl disulfide. Biochim. Biophys. Acta. 1078: 219-25.

23. Lau, B. H.; Lam, F. and Wang Cheng, R (1987): Effect of odor-modified garlic preparation on blood lipids. Nutr. Res. 7: 139-149.

24. Mathew, P. T. and Augusti, K. T. (1973): Studies on the effect of allicin (diallyl disulphide oxide) on alloxan diabetes: Part I—hypoglycemic action and enhancement of serum insulin effect and glycogen synthesis. Indian J. Biochem. Biophys. 10: 209.

25. Miller, G. J; Martin, J. C; Webster. J; Wilkes, H; Miller, N. E; Wilkinson, W. H. and Meade, T. W. (1986): Association between dietary fat intake and plasma factor VII coagulant activity: a predictor of cardiovascular mortality. Atherosclerosis 60: 269-77.

26. Nagai, K. and Osawa, S. (1974): Cholesterol lowering effect of aged garlic extract in rats. Basic Pharmacol. Therapeut. 41.

27. Nakagawa, S; Masamoto, K; Sumiyoshi, H; Kumihiro, K. and Fuwa, T. (1980). Effect of raw and extracted-aged garlic juice on growth of young rats and their organs after peroral administration. J. Toxicol. Sci. 5: 91-112.

28. Papageorgiou, G; Corbet, J. P; Menazes—Brando, F; Pecegueiro, M. and Benezra, C. (1983): Allergic contact dermatitis to garlic (Allium sativum L). Identification of the allergens, the role of mono-, di- and trisulfides present in garlic. A comparative study in man and animal (guinea pig). Arch. Dermatol. Resl 275: 229.

29. Qureshi, A. A; Din, Z. Z; Abuirmeileh, N; Burger, W. C; Ahmed, Y. and Elson, C. E. (1983 a): Suppression of avian hepatic lipid metabolism by solvent extracts of garlic: impact on serum lipids. J. Nutr. 113: 1746-55.

30. Qureshi, A. A; Abuirmeileh, N; Din, Z. Z; Elson, C. E. and Burger, W. C. (1983 b): Inhibition of cholesterol and fatty acid biosynthesis in liver enzymes and chicken hepatocytes by polar fractions of garlic. Lipids 18: 343-8.

31. Rao, A. V. and Ramakrishnan, S. (1975): Indirect assessment of hydroxymethol-glutaryl CoA reductase (NADPH) activity in liver tissue, Clin. Chem. 21: 1523.

32. Sainani, G. S; Desai, D. B; Gorhe, N. H; Natu, S. M; Pise, D. V. and Sainani P. G. (1979): Effect of dietary garlic and onion on serum lipid profile in Jain community. Indian J. Med. Res. 69: 776-80.

33. Sodimu, O; Joseph, P. K. and Augusti, K. T. (1984): Certain biochemical effects of garlic oil on rats maintained on high fat-high cholesterol diet. Experientia 40:78-80.

34. Stamler, J. and Shekelle, R. B. (1988): Dietary cholesterol and human coronary heart disease—the epidemiologic evidence. Arch Pathol. Lab. Med. 112: 1032-40.

35. Thompson, G. R; Soutar, A. K. and Spengel, F. A, Jadhav, A., Gavigan, S. J. and Myant, N. B. (1981): Defects of receptor-mediated low density lipoprotein catabolism in homozygous familial hypercholesterolemia and hypothyroidism in vivo. Proc. Natl. Acad. Sci; USA. 78: 2591-5.

36. Utermann, G. (1994). Lipoprotein (a). In. The metabolic and molecular basis of inherited disease. Eds. In C. R. Scriver, A. L. Beaudet; W. S. Sly, J. B. Stam J. B. Wyngarden and D. S. Fredrickson. McGraw Hill Inc. New York, p. 1887-1912.

37. Prevention in Childhood and youth of adult cardiovascular diseases; time for action. Report of a WHO Expert Committee (1990). World Health Organization Tech. Rep. Ser. 792:1-105.

38. van Ketel W G, de Haan P. (1978): Occupational eczema from garlic and onion. Contact Dermatitis 4: 53-4.

What is claimed is:

1. A method of preparing a blood cholesterol-lowering extract from garlic comprising:
a) contacting crushed garlic with an alcohol to create a first mixture having a solid phase and a liquid phase;
b) separating said solid phase of said first mixture from said liquid phase of said first mixture;
c) contacting, at least once, said separated liquid phase of said first mixture with chloroform to create a second mixture having an alcohol phase and a chloroform phase;
d) separating said chloroform phase of said second mixture from said alcohol phase of said second mixture;
e) concentrating said chloroform phase of said second mixture; and
f) purifying said concentrated chloroform phase by chromatography and obtaining a light yellow product.

2. The method of claim 1, wherein said alcohol is ethanol.

3. The method of claim 1, wherein said purification step uses an eluent having a composition of 2:1 v/v chloroform/methanol.

4. The method of claim 1, wherein said separating in step b) is filtering.

5. The method of claim 1, wherein said separating in step d) is with the use of a separation funnel.

6. The method of claim 1, wherein said purifying is by using silica gel.

7. The method of claim 6, wherein said purifying is by using a silica gel plate.

8. The method of claim 6, wherein said purifying is by using a silica gel column.

9. A method of treating a mammal with a high blood cholesterol level comprising:
a) identifying a mammal in need of such treatment;
b) providing a garlic extract obtained according to claim 1; and
c) administering said garlic extract to said mammal.

10. The method of claim 9, wherein said mammal is a human.

11. A pharmaceutical composition comprising a garlic extract obtained according to claim 1, or a pharmaceutically acceptable salt thereof, and a pharmaceutically acceptable diluent, excipient, stabilizer or carrier.
